(19) United States
(12) Patent Application Publication
(10) Pub. No.: US 2023/0390529 A1
(43) Pub. Date: Dec. 7, 2023

(54) CATHETER, CATHETER SYSTEM AND PROCESSING SYSTEM FOR DETERMINING PARAMETERS DURING A TISSUE PUNCTURING PROCESS

(71) Applicant: KONINKLIJKE PHILIPS N.V., EINDHOVEN (NL)

(72) Inventors: Yitzhack SCHWARTZ, CAESARIA (IL); Zalman IBRAGIMOV, CAESARIA (IL); Mike BERRY, EINDHOVEN (NL)

(21) Appl. No.: 18/032,112

(22) PCT Filed: Oct. 29, 2021
(86) PCT No.: PCT/EP2021/080071

§ 371 Date: Apr. 14, 2023

(30) Foreign Application Priority Data
Oct. 29, 2020 (EP) 20204631.4

(51) Int. Cl. A61M 25/06; A61M 25/01; A61M 25/00; A61B 5/00

(52) U.S. Cl. CPC A61M 25/06; A61M 25/0105; A61M 25/0043; A61M 25/0082; A61B 5/72; A61B 2017/00243

(57) ABSTRACT

A catheter, a catheter system, and a processing system including said catheter or catheter system for determining one or more parameters of tissue of an anatomical structure within a region of interest. The catheter comprising at least two electrodes. The electrical responses of the least two electrodes mounted on the catheter with mutually fixed electrode distance are monitored, and used to derive the parameters.

### FIELD OF THE INVENTION

The invention relates to the field of medical catheters and methods for their use in tissue puncturing procedures, and in particular to cardiac tissue puncturing procedures.

### BACKGROUND OF THE INVENTION

Various cardiac therapy procedures performed on the left side of the heart, such as e.g pulmonary vein (PV) ablation, left atrial appendage occlusion and percutaneous mitral valve repair typically are performed using a transseptal puncturing (TP) procedure to access the left atrium (LA) of the heart from the right atrium (RA).

In a TP procedure a puncturing system is used to create a channel or hole in the fossa ovalis (FO) located in the Inter Atrial Septum (IAS) between the right and left atria. Catheters and delivery systems can be placed through this channel or hole to safely cross the IAS and be introduced into the LA. Accessing the LA without the use of a TP procedure is a fairly difficult or cumbersome process. Other tissue puncturing processes also exist.

There is therefore a desire to improve the performance of a tissue puncturing process, and, in particular to improve that of a TP process. Successful performance of a puncturing process is directly dependent upon the accuracy, validity, and appropriateness of information about the puncturing systems and device used and/or the surrounding tissue.

TP procedures are currently being carried out under fluoroscopy (X-ray) and/or ultrasound (in the case of perforating the IAS, by either trans-esophageal or intra-cardiac echocardiography) guidance. It would be advantageous to improve the determination of information about the TP process, e.g. information about tissue surrounding a puncturing device without the use of such X-ray or US based technologies.

### SUMMARY OF THE INVENTION

While electrical tracking of parts of puncturing devices has been described in the art in e.g. the Chinese patent application CN210158675 U and in Journal Interventional Cardiac Electrophysiology (2011) volume 30 pages 37 to 44, the inventors have recognized that some aspects of the procedure such as the imaging of a structure to be punctured or its' imaging during the puncturing still would require the use of X-ray and/or US techniques or other devices not typically part of the puncturing device therewith complicating the performance of the procedure.

The present disclosure recognizes that a complete puncturing procedure performed without use of X-ray or US based imaging as well as other imaging modalities may be enabled by a specially designed catheter or catheter system for use in such puncturing procedure.

The invention is defined by the claims and provides for catheters, catheter systems, as well as their use with processing systems and medical systems configured to communicate with such catheters and catheters systems as well as methods, optionally embodied in computer programs, for operating such systems.

The present disclosure proposes mechanisms and concepts for use of dielectric-based sensing and/or dielectric based anatomical imaging during a puncturing (also sometimes referred to as penetration) process in general and a TP process in particular. Proposed approaches can advantageously avoid the need for performing fluoroscopy and/or ultrasound imaging during a puncturing process, thereby reducing the adverse effects of the same on a patient and/or reducing cost of procedure as well as time of procedure.

In particular, the present disclosure proposes to treat electrodes of a catheter or puncturing device as internal electrodes for detecting electric fields within an anatomical structure. The electric fields are artificially generated electric fields (i.e. not electric fields generated by the biological entity under investigation) by application e.g. using external body electrodes. The electrical response(s) of the electrodes can be used to derive or measure one or more parameters of the catheter or puncturing device and/or of the surrounding tissue of the anatomical structure.

According to a first aspect of the disclosure there is provided a catheter (90) for use (configured to be used) in a tissue puncturing procedure. The catheter has a tubular body with an axial length, a distal end and a lumen extending from an opening at the distal end along at least part of the axial length. The catheter may have a proximal end with a further opening also giving access to the lumen. The lumen is configured to slidably house a tissue puncturing device such that the tissue puncturing device can be extended outside of the catheter through the opening and beyond the distal end of the catheter or be retracted within the catheter through the opening by a user. Typical examples of tissue puncturing devices are sharp tip needles as known in the art or needles with an electrode (unipolar) at their tip to provide electrical energy supported puncture of tissue. The catheter comprises a first electrode and a second electrode on the distal end where the first electrode is spaced apart from the second electrode with a fixed electrode distance. For example, both electrodes may be fixatedly mounted with respect to each other. They may be fixatedly mounted on the catheter distal end.

The catheter may include electrical signal transmission means for transmitting sensed electrical signals to a proximal end of the catheter at which end the transmission means may be connected to connectors for connecting the electrodes to a processing system as e.g. defined herein.

When the catheter and electrodes are in use and located within a region of interest of a subject to which an electric field is applied, the first and second electrodes are capable of sensing electrical signals caused by the electrical fields applied. The electrode signals may be used by a processing system to determine the desired parameters of devices and/or tissue.

Having electrodes with fixed distances at the end of the puncturing device provides the advantages that it may be used for anatomical imaging of tissue structures to be punctured such as for example the RA or parts thereof such as the IAS or the FO. The anatomical imaging requires a translation of electrical parameters sensed by the electrodes and representative of the applied electric fields to positional coordinates as further explained herein below. Typically, a calibration of the electrodes is performed for this purpose. Electrodes with non-fixed mutual distances are less suitable and difficult to calibrate. Further advantages are described hereinbelow.

In some examples the tubular body includes a tapered portion at the distal end and at least one of the first and second electrodes may be positioned within the tapered portion. There may be one electrode (e.g. the first electrode) at the tip of the tapered portion. The tapered portion may be for dilating a puncture hole in the tissue. In some examples the catheter comprises or is a dilator having the tapered portion. Having electrodes on such tapered portion is advantageous as now the catheter with tapered portion can be used not only for anatomical imaging but can also be used as sensors to be tracked in the applied field to determine its location and/or orientation with respect to an anatomical image of the tissue.

In some examples of catheters there may be more than two electrodes at the distal end each one being spaced apart from at least one other electrode of the ones at the distal end. More fixed distances may provide improved mapping and more accurate location or orientation determinations.

Electrodes (such as at least the first and second electrodes) may be spaced apart along at least the axial length direction. This allows anatomical imaging and axial location and bending (pitch and yaw) determination of the catheter. In some examples the electrodes may be additionally or alternatively spaced apart in one or two non-parallel lateral directions. There may be also electrodes spaced apart in axial direction while others are spaced apart in one or two non-parallel lateral directions. Laterally spaced apart electrodes allow roll orientation about the axial direction.

In some examples of a catheter any or some electrodes are cylindrical electrodes with their cylinder axes parallel to the longitudinal axis of the catheter. At least one electrode dimension such as e.g. the with or length may be for example between 0.5 and 3 mm.

In some examples the catheter comprises means configured to steer at least the distal end of the catheter. Such means may include pull wires and a handheld wire manipulation system as they are known in the art. Steering may include deflection of the tip in one or two directions as known in the art. Steering is not only useful for navigation of the distal end of the catheter to a particular location near an anatomical structure, but is also useful for anatomical imaging which may require probing of the electric field at multiple locations in an anatomical cavity explored.

The current disclosure also provides for a catheter system comprising a catheter as defined herein and at least one of: an introducer catheter and a tissue puncturing device. The introducer catheter has a tubular member with a cavity therein extending between an opening at its distal end and an opening at a proximal end. The cavity is configured to slidably house the catheter as defined herein. The tissue puncturing device includes a distal tip operable as a third electrode.

The system may represent an assembled configuration in which catheter is situated within the introducer catheter and/or a tissue puncturing device is situated within the catheter. However, such system may also have its components as separate parts of a kit of parts or in yet another configuration.

The introducer catheter may include electrical signal transmission means for transmitting sensed electrical signals to a proximal end of the catheter at which end the transmission means may be connected to connectors for connecting the electrodes to a processing system as e.g. defined herein.

In some examples of an introducer catheter there are present means for steering at least the distal end of the introducer catheter. Such means may be the same as described herein above for the catheter.

In some examples of catheter systems, the introducer catheter comprises further electrodes with at least two electrodes being spaced apart with a fixed distance. For example, the introducer catheter may include: a fourth electrode and a fifth electrode on its distal end such that the fourth electrode is spaced apart from the fifth electrode with a fixed electrode distance. As explained herein before for the electrodes on the catheter the electrodes on the introducer catheter can alternatively or additionally be used for anatomical imaging and/or location and orientation detection when it is in use. It may be determined whether a catheter such as dilator is located within an introducer catheter or extended from the introducer catheter. Note that in general catheters are made of plastic materials capable of shielding electric fields.

In some examples of a catheter as defined herein above, the catheter is an introducer catheter having a tubular member with a cavity therein extending between an opening at its distal end and an opening at its proximal end, the cavity being configured to slidably house a further catheter, such as for example a dilator. In these examples, the introducer catheter comprises the first and second electrodes on the distal end. These examples provide the advantages with anatomical imaging and location and orientation tracking without the need for use of a dilator with electrodes. After all, an electrode needle as defined herein before can be used to locate a tip of the dilator when the electrode is quite near the tip and just outside of the dilator.

The introducer catheter may comprise steering means configured to steer at least the distal end as described herein before for the catheter. Steering is useful for the anatomical imaging and/or navigation of the catheter by a user.

Some catheter systems as defined herein comprise the introducer catheter having the fixed electrode spaced electrodes and at least one of: a dilator without electrodes and a tissue puncturing device. The dilator includes a tubular body having an axial length and a distal end and a dilator lumen extending from an opening at the distal end along at least part of the axial length. The dilator lumen is configured to slidably house a tissue puncturing device (120) such that the tissue puncturing device can be extended outside of the dilator and beyond the distal end through the opening or be retracted within the dilator by a user. The tissue puncturing device includes a distal tip operable as a third electrode.

In some examples a catheter or introducer catheter comprises an anchoring structure at the distal end of the catheter which anchoring structure is reversibly extendible from the catheter or introducer catheter and which anchoring structure comprises at least one further electrode. The anchoring structure may be used to anchor the catheter or introducer catheter to an anatomical structure. For example, it may be anchored to the FO of an IAS. There exist transseptal puncturing catheter systems that have a looped wire as an anchoring structure for anchoring to the FO. The anchoring structure provides increased positional and/or orientational stability of the distal end of the catheter or catheter system during a procedure. Having an electrode as a positional sensor is advantageous. The loop may be made of an electrically conductive material (e.g. metal) that is electrically addressable.

The disclosure provides for a processing system for determining one or more parameters of tissue of an anatomical structure within a region of interest, and optionally one or more parameters of the catheter, using electrical responses received or obtained from electrodes of a catheter and/or introducer catheter as defined herein. The processing system comprises: an interface connectable to two or more electrodes of a catheter such as for example at least first and second, or third and fourth electrodes, of the catheter and/or introducer catheter. The interface is configured to receive or obtain electrical responses from the electrodes such as at least: first electrical responses representative of electric fields induced within the region of interest; and second electrical responses to the electric fields, wherein the first and second electrical responses are respectively received or obtained from the first and second electrodes respectively, or the fourth and fifth electrodes respectively. The processing system further comprises a processor circuit in communication with the interface and configured to determine the one or more parameters based on at least the first and second electrical responses. The processing system optionally comprises an output configured to output data representative of the one or more parameters of the puncturing device and/or surrounding tissue of the anatomical structure based on at least the first and second electrical responses. The output is communicatively coupled to the processing system.

The output may be configured for coupling to a user interface such as for example a display device

In some examples of a processing system the first electrical responses comprise first and third electrical responses representative of electric fields induced within the region of interest and the second electrical responses comprise second and fourth responses representative of electric fields induced within the region of interest, wherein the first and second electrical responses are received or obtained from the first and second electrodes, respectively, and the fourth and fifth electrical responses are received or obtained from the fourth and fifth electrodes respectively. The processor circuit is then configured to determine the one or more parameters of the puncturing device and/or surrounding tissue of the anatomical structure based on at least the first, second, fourth and fifth electrical responses. In such case electrode responses of multiple electrodes with multiple fixed distances are processed into the parameters and this may provide improved quality of parameters.

In some examples the one or more parameters comprises an anatomical representation of the anatomical structure. The representation may be an image of an anatomical structure. The representation can be a model of the anatomical structure in a coordinate system. Additionally, the parameters may comprise tracking data of a catheter and, optionally, puncturing device. The tracking data may be coordinates of electrodes of the catheter in an image of the coordinate system in which an anatomical representation is present.

The processor circuit can be further configured to receive from a look-up table calibration data comprising for each fixed electrode distance one or more correlations between the fixed electrode distance and parameters representing electric fields measured at the electrodes spaced by the fixed electrode distances. The processor circuit can be further configured to determine the anatomical representation of the anatomical structure from the first and second data using the calibration data.

The disclosure further provides a computer-implemented method for determining one or more parameters of tissue of an anatomical structure within a region of interest using electrical responses received or obtained from electrodes of a catheter or introducer catheter as defined herein. The computer-implemented method comprises the step of: receiving or obtaining, by a processing system: first electrical responses representative of electric fields induced within the region of interest; and second electrical responses to the electric fields, wherein the first and second electrical responses are respectively received or obtained from the first and second electrodes respectively, or the fourth and fifth electrodes respectively. The method further comprises determining, by a processor circuit of the processing system, the one or more parameters of tissue of an anatomical structure within a region of interest based on the first and second electrical responses. The method further includes the optional step of providing data representative of the one or more parameters of tissue of the anatomical structure based on at least the first and second electrical responses.

In some examples of the computer implemented method the one or more parameters comprises features as described for the system described herein before and after.

The computer implemented method may be further defined such that the processor circuit is further configured to perform the steps to receive from a look-up table calibration data comprising for each fixed electrode distance one or more correlations between the fixed electrode distance and parameters representing electric fields measured at the electrodes spaced by the fixed electrode distances. In such case the step of the determining of the one or more parameters of tissue of an anatomical structure within a region of interest is further based on the calibration data.

The disclosure provides for a computer program product comprising instructions which, when executed by a processing system, cause the processing system to carry out the computer implemented methods as defined herein. The computer program product may be stored on a memory device, non-transitory storage medium or computer network or other means. Such medium may be read by the processor circuit.

The now following examples can be used in conjunction with other aspects and examples described, or alternatively, they can be independent from aspects and examples described herein before. Thus, the disclosure also provides for examples of a processing system for determining one or more parameters of a puncturing device, comprising a sheath that houses a needle having a distal end for penetrating tissue of an anatomical structure, and/or parameters of surrounding tissue of the anatomical structure.

The processing system comprises: an input interface configured to obtain: at a first input, and from the distal end of the needle, a first set of one or more electrical responses of the distal end of the needle to one or more electrical fields induced in the anatomical structure, wherein the distal end of the needle is formed of a material responsive to changes in an electric field; an at a second input, and from a first electrode mounted on the sheath, a second set of or more electrical responses of the first electrode to one or more electrical fields induced in the anatomical structure; and at a third input, and from a second electrode mounted on the sheath, a third set of one or more electrical responses of the second electrode to one or more electrical fields induced in the anatomical structure; and a processor circuit configured to determine one or more parameters of the puncturing device and/or surrounding tissue of the anatomical structure using at least the first set of one or more electrical responses, the second set of one or more electrical responses and the third set of one or more electrical responses.

The present disclosure has recognized that the distal end of a puncturing device can advantageously be treated as an electrode for monitoring electrical fields induced in the anatomical structure. This facilitates use of the distal end of the penetrating for determining characteristics of the puncturing device and/or the surrounding tissue.

In particular, it is recognized that the combination of electrical responses of a distal end of the needle and two electrodes on a sheath of the puncturing device can provide useful information for aiding in the performance of a penetrating process, such as a transseptal procedure. The use of two sheath-based electrodes advantageously allows for tracking of the sheath (and, in particular, a direction in which a needle housed in the sheath faces, even when completely covered by the sheath), whilst using the needle as an electrode allows for the position of the needle to be tracked when it exits the sheath (with the position being definable using the sheath electrode(s)).

For example, the electrical response(s) could be tracked to monitor a location of the distal end and/or the sheath of the penetrating device. This could aid in ensuring that the puncturing device is maneuvered to an appropriate location and that appropriate tissue is punctured.

In a particularly advantageous example, the electrical responses of the electrodes mounted on the sheath could be tracked to perform an imaging process of the anatomical structure, to thereby construct an anatomical model of the anatomical structure. This means that a same device can be used to perform both mapping of the anatomical structure and to perform the puncturing, reducing the number of intrusive objects that need to be inserted into the individual, thereby reducing exposure to potential infection or cross-contamination.

As another example, the electrical response(s) could be tracked to derive a thickness of tissue in the vicinity of the distal end, thereby facilitating ease in tracking an appropriate location for penetrating the tissue (e.g. for improved ease in creating a through hole in the tissue). As yet another example, the electrical response(s) may be processed to identify when the distal end makes contact with tissue, thereby facilitating ease in tracking a puncturing process.

Suitable examples of material responsive to changes in an electric field may include medical-grade metals, such as titanium, (surgical) stainless steel, or cobalt-chrome alloy; as well as other suitable materials. The material responsive to changes (for the distal end of the needle) should be sufficiently sturdy to be able to puncture tissue.

The distal end of the needle is designed for puncturing tissue, e.g. formed of a suitably shaped and/or sharp structure for puncturing tissue.

The needle is housed in a sheath, e.g. a largely cylindrical element through which the needle is threaded. The sheath can act to protect tissue surrounding the needle from its distal end until a desired location or zone for performing a penetrating process with the puncturing device is reached.

By monitoring one or more parameters of the surrounding tissue and/or the sheath using the electrode mounted on the sheath, additional information for use in a puncturing process can be obtained, e.g. the relative location of the sheath during a puncturing process. This can facilitate identification of the location of the puncturing device, e.g. during transit to the desired location, and/or be used to determine characteristics for surrounding tissue in tandem with the distal end of the needle.

The present disclosure thereby facilitates a mechanism for determining clinically useful characteristics of tissue and/or the puncturing device.

The processing system may further comprise an output interface configured to provide, to a further device, an output signal responsive to the determined one or more parameters of the puncturing device and/or surrounding tissue of the anatomical structure. The further device may, for example, comprise a user interface, a memory, a further processing system, an alerting system or any other suitable further device for receiving an output signal.

The processing system may be further configured to display, at a user interface, a visual representation of the determined one or more parameters of the puncturing device and/or surrounding tissue. Displaying a visual representation of the determined parameters can provide a clinician, e.g. controlling a puncturing operation of the puncturing device, with useful clinical information to aid their control of the puncturing device to achieve a desired medical goal, e.g. puncturing or puncturing of tissue. This information can thereby credibly assist a clinician in the performance of a technical task.

The one or more parameters of the puncturing device and/or surrounding tissue may comprise a relative position of the sheath and/or the needle with respect to the anatomical structure. Determining a location of the puncturing device is clinically helpful in ensuring that the correction puncturing location for the tissue has been identified, and to mark (e.g. for subsequent use) a location of the puncturing performed by the puncturing device. This proposed disclosure thereby facilitates knowledge over a position of a puncturing performed by a puncturing device.

In some preferred embodiments, the one or more parameters of the puncturing device and/or surrounding tissue comprises an orientation/direction of the sheath and/or the needle. This increases an ease of tracking the movement of the needle during the transseptal procedure, e.g. to ensure that the needle is moving in the correction direction for performing the transseptal puncture.

In some embodiments, the input interface is further configured to obtain, at a fourth input and from an modelling system or memory, an anatomical model of the anatomical structure, and the one or more parameters of the puncturing device and/or surrounding tissue comprises a relative location of the sheath and/or needle with respect to the anatomical model of the anatomical structure.

The skilled person will appreciate that determining a relative location of the puncturing device with respect to the anatomical model is effectively equivalent to determining a relative location of the puncturing device with respect to the anatomical structure, as the anatomical model is a description of the anatomical structure in encoded form or computer language.

In some examples, the anatomical model of the anatomical structure is generated by monitoring the location of one or more electrodes mounted upon an intraluminal device. In this way, the position of the distal end of the needle can be derived in a same virtual space as used to generate the anatomical model of the anatomical structure. This increases an ease in determining a relative location of the distal end to the anatomical model.

The processing system may be adapted wherein the first or second electrode comprises an anchoring structure configured to secure or couple the sheath to tissue during a penetrating procedure of the puncturing device.

Some sheaths for puncturing devices may comprise an anchoring structure that connects or anchors the sheath to tissue during a puncturing process (e.g. to reduce a movement of the sheath and/or puncturing device during the puncturing process). The anchoring structure may also be used to apply tension to the tissue to aid in puncturing the tissue with the puncturing device.

The present disclosure recognizes that this anchoring structure could be further repurposed for use as an electrode, and can thereby provide additional information (in the form of electrical responses) for determining one or more parameters of the puncturing device. This additional information can be clinically useful. This also reduces the number of electrodes that need to be positioned on the sheath, e.g. if the sheath only comprises the anchoring structure and one other sheath electrode.

In some examples, the anchoring structure is moveable relative to other components of the sheath. The anchoring structure may thereby be used, for example, to brace the sheath against the tissue or to push tissue away from the sheath. The anchoring structure may comprise a loop of material, e.g. a loop of (medical-grade) metal. The movement of the sheath may follow predefined mechanical properties.

There is also proposed a medical system for penetrating tissue of a subject, the medical system comprising: a puncturing device comprising: a needle having a distal end for penetrating tissue of an anatomical structure, the distal end of the needle being formed of a first material responsive to changes in an electric field; a sheath that houses the needle; an electrode mounted on the sheath; and the processing system herein disclosed.

The sheath and the needle may be separable or inseparable elements. Thus, the sheath and the needle may form a kit of parts that, when together, form the puncturing device.

In some examples, the distal end of the needle is configured to puncture the inter-atrial septum of a subject. In other words, the puncturing device may be designed for a transseptal procedure.

The medical system may comprise one or more external electrodes, to be positioned externally to the subject, controllable to generate one or more electric fields, wherein the distal end of the needle is configured to be responsive to changes in the one or more electric fields generated by the one or more external electrodes. In some examples, the processing system may control the operation of the external electrodes, e.g. control an electric field generated by the external electrodes.

There is also proposed a computer-implemented method for determining one or more parameters of a puncturing device, comprising a sheath that houses a needle having a distal end for penetrating tissue of an anatomical structure, and/or surrounding tissue of the anatomical structure.

The computer-implemented method comprises: obtaining, from the distal end of the needle, a first set of one or more electrical responses of the distal end of the needle to one or more electrical fields induced in the anatomical structure, wherein the distal end of the needle is formed of a material responsive to changes in an electric field; obtaining, from a first electrode mounted on the sheath, a second set of or more electrical responses of the first electrode to one or more electrical fields induced in the anatomical structure; and determining one or more parameters of the puncturing device and/or surrounding tissue of the anatomical structure using at least the first set of one or more electrical responses, the second set of one or more electrical responses and the third set of one or more electrical responses.

The computer-implemented method may further comprise providing, to a further device, an output signal responsive to the determined one or more parameters of the puncturing device and/or surrounding tissue of the anatomical structure.

The one or more parameters of the puncturing device and/or surrounding tissue may comprise a relative position of the distal end of the needle with respect to the anatomical structure.

There is also proposed a computer program product comprising code which, when executed by a processor circuit, causes the processor circuit to perform the steps of any herein described method. There is also proposed a non-transitory computer-readable medium or data carrier comprising or carrying the computer program product.

The present disclosure also proposes a computer program (product) comprising instructions which, when the program is executed by a computer or processing system, cause the computer or processing system to carry out (the steps of) any herein described method. The computer program (product) may be stored on a non-transitory computer readable medium.

Similarly, there is also proposed a computer-readable (storage) medium comprising instructions which, when executed by a computer or processing system, cause the computer or processing system to carry out (the steps of) any herein described method. There is also proposed computer-readable data carrier having stored there on the computer program (product) previously described. There is also proposed a data carrier signal carrying the computer program (product) previously described.

The skilled person would be readily capable of adapting any herein described method to reflect embodiments of herein described apparatus, systems and/or processors, and vice versa. A similar understanding would be made by the skilled person with respect to a computer program (product).

Additional aspects, features, and advantages of the present disclosure will become apparent from the following detailed description.

### BRIEF DESCRIPTION OF THE DRAWINGS

For a better understanding of the invention, and to show more clearly how it may be carried into effect, reference will now be made, by way of example only, to the accompanying schematic drawings, which are not to scale, and in which:

FIGS. 1A to 1C illustrate cross sections of distal ends of catheters and catheter systems (e.g. puncturing systems) as defined herein;

FIG. 1D illustrates a catheter system having a looped portion.

FIG. 2 illustrates a medical system having a processing system;

FIG. 3 illustrates a point cloud to surface reconstruction procedure;

FIGS. 4 to 6 illustrate a puncturing process;

FIGS. 7 and 8 provide a representation of an anatomical model;

FIG. 9 provides a flowchart according to an embodiment; and

FIG. 10 illustrates a processor circuit.

### DETAILED DESCRIPTION OF THE EMBODIMENTS

Aspects and examples of the current disclosure will be described with reference to the FIGS. The detailed description and specific examples, while indicating exemplary embodiments of the apparatus, systems and methods, are intended for purposes of illustration only and are not intended to limit the scope of the invention. These and other features, aspects, and advantages of the apparatus, systems and methods of the present disclosure will become better understood from the following description, appended claims, and accompanying drawings. The same reference numerals are used throughout the figures to indicate the same or similar parts.

The disclosure provides a mechanism for determining (values of) one or more parameters of a catheter system (used with a puncturing device) and/or surrounding anatomical tissue. The electrical responses of at least two electrodes mounted on a catheter or sheath of the catheter system (e.g. puncturing system) and, optionally a distal end of a needle of the catheter system puncturing system are monitored, and used to derive the parameters. Such electrode responses may be monitored using a processing system configured to receive the responses. The processing system in turn may be part of a medical system such as a dielectric imaging system, an electro-anatomical mapping system or the like. Operation of such systems will be described hereinafter in some detail, but it is noted that such systems are known per se for example from U.S. Pat. Nos. 5,697,377, 5,983,126, WO2018130974 and WO2019034944.

Embodiments of the disclosure thereby provide useful information for a clinician to perform a tissue penetrating process, such as for example transseptal puncturing process. Thus, the location of important elements of the puncturing device may be tracked. For example, the location may be tracked with respect to a tissue of a region of interest of a subject such as a wall of a lumen of an anatomy of the subject. A model or representation of the tissue may additionally or alternatively be provided. The present disclosure also recognizes that certain elements of the puncturing system or device can be adapted for use as intra-body or internal electrodes, i.e. can have a dual purpose. In such instance it has also been recognized that some characteristics of the electrodes also need to be taken into account when providing the different parameters.

The systems, devices and methods disclosed herein are not limited to transseptal puncturing perse. They may be used for medical procedures aimed at treatment of the blood circulation system. These would include but are not limited to: structural repair, such as with structural heart disease or aneurism repair, and treatment of arrythmia for example of the atrium.

In the context of the present disclosure, the term “internal” refers to within the body of a subject, and the term “external” generally refers to outside of the body of the subject. The term “determine a parameter” means to determine, predict, measure or otherwise define a value or values for a particular parameter.

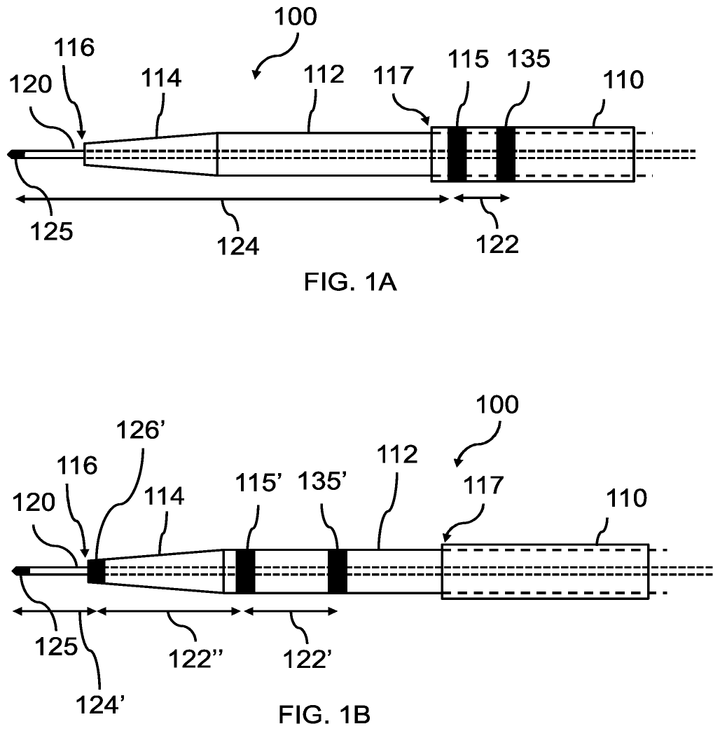

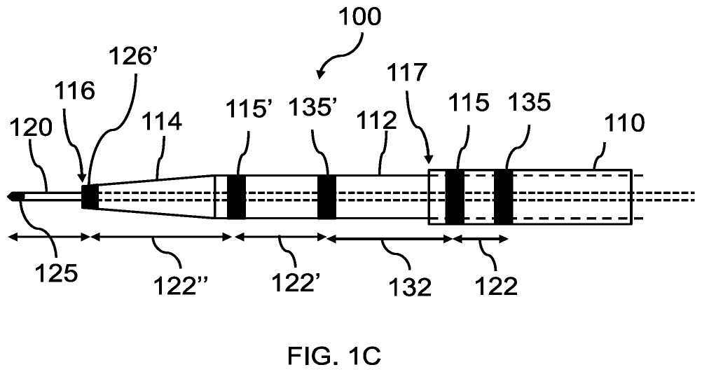

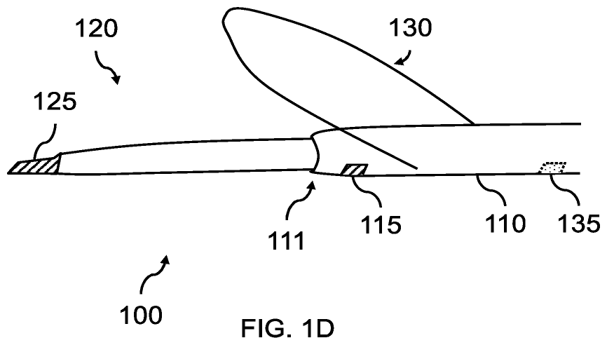

FIG. 1A illustrates a distal end of a first example of a catheter system 100. The catheter system includes a cylindrically shaped sheath 110 having a sheath lumen along its length direction (axial axis of the sheath) from the tip 117 of the sheath lumen to an opening at a proximal end of the sheath (not shown). The sheath in this case is part of an introducer catheter. The lumen is configured to house a cylindrical dilator 112 having a tapered end 114 with an opening 116 at its distal tip giving access to a dilator lumen extending along a length direction (along the cylinder axis) of the dilator to a proximal end of the dilator (not shown). The sheath lumen is configured to slidably house the dilator 112 such that the dilator may be extended out of or retracted within the sheath lumen through the distal end opening at the tip 117. In some embodiments the dilater is fixed and not slidable. The dilator in such case may be part of the sheath or integrated within the sheath. However, in the currently shown example it is slidably contained within the sheath lumen. The sliding of the dilator may be controlled by a user form a proximal end of the catheter system which is not shown for clarity and generally known in the art.

In some embodiments, such as the one shown, the catheter system also includes a needle 120 which is slidably contained within the dilator lumen such that the needle may be extended out of or retracted within the dilator lumen through the opening 116 of the dilator. The needle is the part of the catheter system that is used to make the first puncture of tissue either by force using its sharp tip or by providing electrical or laser energy from its tip or distal end 125.

In some embodiments, the dilator and needle are also rotatable about the cylinder axis within the lumen they are respectively housed in.

The catheter system has a proximal end comprising the proximal ends of the sheath the dilator and the needle. The proximal end comprises means as known in the art for the slidable, and rotatable if applicable, manipulation of the dilator and needle by a user. For example, there may be handheld controls and locks and grips for such purpose. If any catheter such as the introducer catheter is steerable then there may be e.g. pull wires as usual in the art.

A device as described is typically used in the following way during a puncturing procedure. The sheath including dilator and needle, with the needle retracted into the dilator) is maneuvered near the to be punctured tissue by insertion into the region of interest. The sheath may be steerable for this purpose as known in the art. For example, the sheath is inserted in the venous system of a subject to be guided to the right atrium via the vena cava. After a desired puncturing location has been found, the needle is extended from the dilator to puncture the tissue. For example, the FO in the IAS from the RA of the heart may be punctured to enter the LA of the heart. Subsequently the dilator is used to enlarge (dilate) the puncture hole such that after dilation the sheath itself can be advanced through the dilated puncture hole. At this point the dilator and needle may be retracted into the sheath and removed entirely from the sheath form the proximal end of the catheter system to leave the sheath extended through the dilated puncture hole to be used for insertion of desired repair tools (other catheters) into the LA of the heart via the sheath lumen.

The needle may be completely removable from the catheter system through the opening of the proximal end of the dilator. This may be advantageous if the sheath is not of the steerable type. In such case the sheath and dilator may be guided using a steerable guide wire housed withing the dilator lumen. Once the sheath and dilator are in place, the guide wire may be replaced with the needle to do the puncturing as described herein before.

Although described as part of the catheter systems in the herein described examples, the needle need not be part of the catheter system to provide the catheter systems with one or more of its beneficial characteristics. For example, an imaging functionality requires no presence of the needle. In such case the catheter system does include the needle, such system may be referred to as a puncturing system.

Since a puncturing procedure typically is a delicate procedure to perform it is beneficial to be able to provide information of the steps of such puncturing process using the catheter system. To this end the catheter system has a plurality of spaced apart electrodes with a well-defined fixed spacing disposed on its distal end with which electrical responses can be collected during a puncturing procedure. The electrodes may be fixed at defined positions on a catheter. How such responses can be evoked will be elucidated herein below. For example, on the sheath 110 of the catheter system of FIG. 1A there are disposed a first electrode 115 and a second electrode 135. The conform to the circumference of the cylindrical sheath and have a constant width measured in the length direction of the sheath. In this case their width is equal. The electrodes are spaced apart in the length direction with a fixed distance df 122. This fixed distance enables the use of the electrodes as a translator (e.g. as a kind of ruler) between electrical signals measured with the electrodes and the distance between locations where such signals were measured. This is beneficial for anatomical imaging purposes of tissue in the vicinity of the electrodes in a way that will be described in more detail herein below. Note that in case the needle tip 125 would also provide an electrode for measuring electrical responses, it would not provide a reliable or accurate translator/ruler together with any one of the first and second electrodes. After all, it is located on the needle such that its distance from the any one of the first and second electrodes (for example distance dv 124) would not be fixed but variable since the needle 120 is slidably movable with respect to the sheath 110.

FIG. 1B shows a second example of a distal end of a catheter system 100. The catheter system includes similar parts as described for FIG. 1A. However, in this case there is a plurality of electrodes mounted on the dilator 112 instead of on the sheath 110 as was the case for the example of FIG. 1A. In particular, the dilator includes a first electrode 115′, a second electrode 135′ and a third electrode 126′ with fixed interelectrode distances 122′ and 122″. As described for the electrodes 115 and 135 of FIG. 1A, these electrodes 115′, 135′ and 126′, provide the advantages for imaging, because of their fixed spacings. Note that as for the distance 124 in FIG. 1A, the interelectrode distances in the example of FIG. 1B measured from a tip electrode 125 to anyone of the others (e.g. distance 124′), do not provide the most reliable or accurate translators/rulers used for dielectric imaging.

FIG. 1C shows yet another distal end of an example catheter system 100. It is a combination of a sheath as described in FIG. 1A and a dilator as described with reference to FIG. 1B. The sheath 110, houses a needle 120 with electrode tip 125. The dilator 112 is configured to be slidably movable within the sheath 120 so that the sheath can cover and uncover the dilator and its electrodes as it moves with respect to the sheath 110. In this example there are two sets of a plurality of electrodes that can be advantageously used for the dielectric imaging; i.e. the set on the dilator and the set on the sheath as each of the sets has fixed well defined distances to be used for that purpose.

Thus, it has been recognized that the catheter system should have at least one of such set of fixed distance electrodes. While such advantage has been described with reference to the use of such electrodes for dielectric imaging, such electrodes may also be of importance to other applications. For example, dielectric sensing or impedance sensing to determine tissue parameters such as e.g thickness, constitution, state (ablated or not) etc. also benefit from the electrode configuration with fixed interelectrode distance. It is noted that for any of these purposes additional electrodes with non-fixed interelectrode distances can be used in combination with the fixed electrodes in various cases. For example, the electrodes may be used for locating the electrodes in a coordinate system. This does not need the fixed distance per se, although the fixed distance may be used to check measured locations on whether they are far off the fixed distance etc.

The catheter system may comprise or consist of a transseptal puncturing catheter system and the needle 120 may, for example, be a transseptal needle for puncturing an intra-atrial septum (IAS). Appropriate transseptal needles with sharp tips, electrode tips or other ablative tips are known perse and will not be further described herein as they are generally known in the art.

In electrode needles the distal end 125 of the needle 120 is configured to be made of a material responsive to changes in an electric field (e.g. in which the needle is placed). In other words, an electrical response (e.g. an electrical signal) of the distal end 125 of the needle 120 may change in response to a change of a parameter (magnitude, frequency etc.) of an electric field into which the needle is positioned. Suitable materials would be apparent to the skilled person and may include, for example, metals (e.g. titanium, (surgical) stainless steel, cobalt-chrome alloy or the like). In this way, the distal end 125 of the needle 120 can be treated as an electrode, e.g. a unipolar electrode.

A sheath, dilator or needle that includes electrodes as described herein comprises electrical signal conductors, and electrical signal outputs interconnected such that an electrical signal responsive to the changes in electric field picked up by any electrode can be transmitted via the signal conductors to the outputs. For example, the sheath, dilator or needle may have wiring electrically connected to the electrode and to a signal output (i.e. in the form of an appropriate connector) at the proximal end of the needle. The needle may be at least partly or entirely made of a conductive material where the conductive material can be used as the conduction means. Alternatively, or additionally, there may be a wireless communication of the signal from the electrode to the output.

Any sheath as disclosed herein may be formed of a biocompatible material such a plastic material as known in the art. There may be a brading for reinforcement as also known in the art.

The sheath and/or dilator may effectively act as a catheter to thread through one or more body lumens and/or cavities (e.g. to reach the right atrium for performing a transseptal puncture). Such devices may have one lumen as described herein, but there may also be more than one per device. Each of the lumen in a sheath and/or dilator may be configured to house different other catheters or guidewires. In any case, the sheath may transport the dilator and/or needle through one or more lumens and/or cavities of the subject.

FIG. 1D shows a further distal end of an example catheter system (puncturing system) 100. As described for the other examples, there is a needle 120 having a tip 125 slidably held by a sheath 110. The sheath has an opening 111 at its distal end through which the needle may be extended or retracted.

The puncturing system further comprises a first electrode 115 mounted on the sheath 110. The first electrode 115 is configured to be responsive to one or more electric fields into which the first electrode is placed. The first electrode 115 may be an immovable electrode with respect to the sheath, i.e. its relative position on the sheath may be fixed.

In one example the system 100 further comprises a second electrode 135 which may be used as described herein before in combination with the first electrode.

In some embodiments or examples, the system may have an anchoring structure 130. The anchoring structure 130 is configured to anchor, secure or couple the sheath to tissue (of a patient) during a penetrating procedure, performed using the needle 120. Thus, the anchoring structure 130 may effectively anchor the sheath to the tissue, e.g. to reduce or prevent movement of the sheath during a penetrating procedure or to aid in deflection/bending of the sheath (to guide the needle into an appropriate position). The anchoring structure may be configured to anchor within the fossa ovalis.

In some embodiments or examples, the anchoring structure 130 is configured to comprise material that is electrically responsive to electric fields into which the anchoring structure is placed. Thus, the anchoring structure can be treated as an electrode, e.g. a unipolar electrode. And as previously explained, the anchoring structure 130 can act as a second electrode 135 mounted on the sheath or yet as a further electrode on the sheath.

The anchoring structure 130 may, for example, comprise a loop of electrically responsive material, such as the anchoring nitinol loop of the TSP Crossee™ transseptal access system (Transseptal Solutions).

Preferably, the anchoring structure 130 may be configured to be controllably movable with respect to the sheath, e.g. to extend out of and/or retract back into the sheath. This means that the anchoring structure can be deployed (e.g. extended outwardly) as the sheath/needle nears, or is at a part of, tissue that is to be punctured/punctured. The illustrated anchoring structure 130 is depicted in an extended position. To this end the sheath may comprise a mechanical arrangement operably connected to the anchoring structure and control knobs at the proximal end of the sheath and operably connected to the mechanical arrangement such that the extension or retraction of the anchoring structure may be controlled via manipulation of the control knobs by a user (subject or further machine).

In some other embodiments, the second electrode may comprise an electrode 135 which is immovable with respect to the sheath, like the first electrode 115. If present, this second electrode 135 is also configured to be responsive to one or more electric fields into which the second electrode is placed. Of course, in such examples, the anchoring structure may still exist, but need not be electrically responsive.

If the anchoring structure 130 is electrically responsive, the electrode 135 may be omitted.

In some embodiments, the puncturing device comprises two sheath electrodes, i.e. electrode 115 and electrode 135, and an anchoring structure comprising an electrode (i.e. three electrodes mounted on the sheath), as well as the needle. This facilitates yet further improved tracking of components of the puncturing device.

Electrodes may be made of a metal. They may have a width measured in the axial direction of between 0.2 to 5 mm Their mutual distances may be between 0.5 to 10 mm A distal end may have a length measured in the axial direction from its tip of less than 15 cm, or less than 10 cm and is preferably less than 7 cm. Diameters of catheters can be chosen as usual in the art albeit that they should be chosen such as to allow them to be housed by other catheters or house other catheters if required.

From the foregoing, it is apparent that in some examples there are at least three possible elements (“electrodes”) of the puncturing device that are responsive to an electric field into which they are placed: the distal end of the needle; the first electrode on the sheath and the second electrode on the sheath, which may be embodied as an anchoring structure for securing the sheath against tissue of the patient.

The present disclosure recognizes that the electrical response(s) of the electrodes to electric fields induced in an anatomical cavity can provide useful information. In particular, a processing system or medical system including such processing system (e.g. a dielectric imaging or mapping system) can exploit these responses to generate additional or more accurate information about the puncturing device and/or the anatomy of the subject to improve a clinician's understanding. While the fixed distance electrodes can provide accurate translation elements (rulers) for the imaging of an anatomy or part thereof or accurate dielectric sensing for other purposes, any of the electrodes, whether having fixed interelectrode distances or not, may be used to determine a location and/or orientation of parts of the puncturing system with respect to an anatomy or part thereof. In particular, the present invention recognizes that these elements are particularly advantageous for use with a puncturing device for transseptal procedures. In particular, this proposed approach allows a same device to be used for both electrical mapping of the anatomical structure (e.g. using the electrodes on the sheath) and for tracking the position, and direction of the needle (e.g. using the electrical responses of the needle and one or more of the responses of the sheath electrodes).

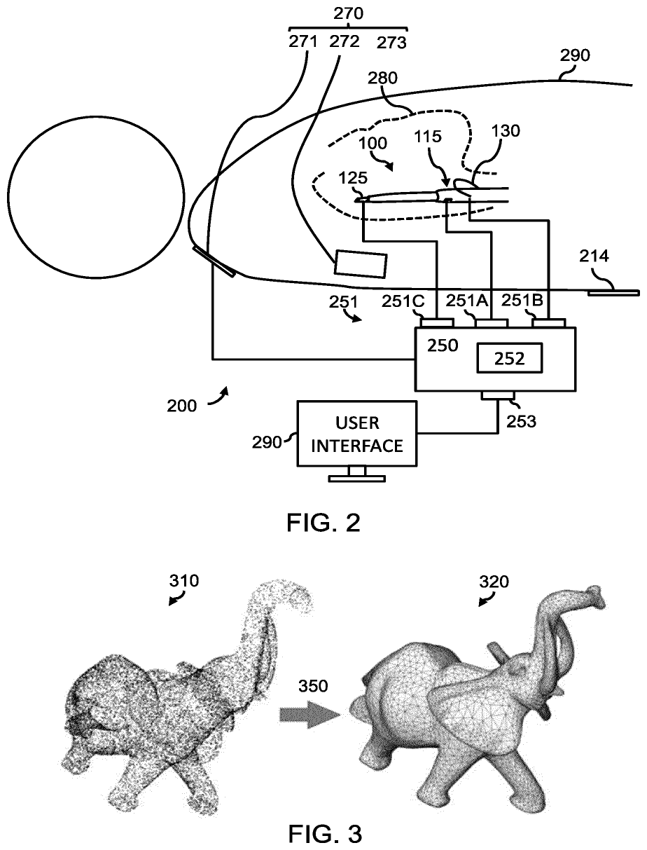

To use the electrical signals provided by the electrodes of a catheter systems as described herein, a processing system is configured to receive them and the parameters of the catheter system or the surrounding tissue. FIG. 2 illustrates an example medical system 200 for penetrating tissue, such as an IAS, of a subject. The medical system can comprise a dielectric imaging system or electro-anatomical mapping system such as for example a Carto™, Kodex™, EnSite™, Rythmia™ or other similar system that is capable of creating an electric field in a region of a subject which electric field may evoke electrical responses in the electrodes of the cather systems disclosed herein when inserted in the region of interest. For example, he region of interest may comprise cavities of the heart in which a catheter is inserted.

The medical system 200 may comprise a medical catheter system (e.g. in the form of puncturing system or device 100) as disclosed herein, such as anyone described with reference to FIGS. 1A to D. In operative state, the catheter system is electrically connected to such catheter system. To such end the medical system includes a processing system 250.

The processing system 250 is configured to be connected to the electrodes via electrical output means of the catheter system and to receive and process the response(s) of the electrodes of the medical catheter system 100 to determine one or more parameters of the medical catheter system and/or surrounding tissue of the anatomical structure using at least the first set of one or more electrical responses and the second set of one or more electrical responses. The processing system 250 and any medical system including such processing system is, by itself, an embodiment of the disclosure.

The medical system 200 may further comprise one or more external electrodes 270, for example in the form of body patch electrodes, to be positioned externally to the subject and controllable to generate one or more electric fields within a region of interest in the subject. Thus, the electrodes are configured to generate the one or more electric fields within an anatomy of the subject located within the region of interest. The electrodes of the puncturing device respond to these electric fields, e.g. they can adopt voltages in accordance with an electric field generated at the location of an electrode. The external electrodes may be controlled by the processing system which may have a suitable interface for connecting any communication leads of the electrodes to and for addressing the electrodes and providing the necessary electrical signals to the electrodes. Alternatively, a separate external electrode controlling system may be used.

The processing system 250 further comprise an input interface 251. The input interface comprises inputs for connecting to the electrodes of the medical catheter systems defined herein. The processing system may receive or obtain electrode responses from the electrodes of the medical catheter systems through this interface. Thus, as an example, the interface of FIG. 2 is shown to include a first input 251A, a second input 251B and a third input 251 C.

The input interface 251 of the processing system may therefore be communicatively coupled to the electrodes of the medical catheter system (e.g. puncturing device). Thus, the processing system 250 is configured to electrically communicate with two or more electrodes on the medical catheter system. In this way, the processing system may effectively treat the electrodes of the catheter system as intra-body or internal body electrodes.

In the FIG. 2, the first input 251A is connected to a first electrode 115 of a medical catheter system 100 and a second input 251B is connected to a second electrode of the medical catheter system 100. Also shown is an optional third input 251C connected to a third electrode 125 located on a needle of the medical catheter system.

The medical catheter system shown has its two electrodes mounted on the sheath connected such that the processing system may receive or obtain a first set of one or more electrical responses to one or more electrical fields induced in the anatomical structure and the processing system may receive or obtain a second set of one or more electrical responses to the one or more electrical fields induced in the anatomical structure. The third input may be used to receive or obtain a third set of electrical responses measured by the needle electrode and responsive to the one or more electrical fields.

The sets of responses may be used in different ways depending on the parameters to be determined from them.

The processing system 250 comprises a processor circuit 252 that is configured to process any signals or data representative of or comprising the sets of electrical responses. To determines one or more parameters of the medical catheter system (e.g puncturing device) and/or surrounding tissue of the anatomical structure based on these sets of electrical responses. The processor circuit 252 of FIG. 2 thus is configured to process first, second and, optionally, third sets of responses. Different combinations of the first, second and, optionally third sets, of responses may be used to determine the different parameters. Example parameters include a location, an orientation and/or a shape of the medical catheter system (e.g. puncturing device), a location of the distal end of the needle, a distance that the projection needle extends out of the puncturing device, a direction and/or orientation of the needle; a map or model of the anatomical structure, a shape of the anatomical structure, a thickness of tissue of the anatomical structure and so on. Such location and orientation parameters may be determined with respect to a model of an anatomy the medical catheter is in or close to and this may entail representing the model and at least a part of the medical catheter system (e.g. the part carrying the electrodes) within a common coordinate system as is usual in the art.

The processing system 250 may further comprise an output interface 253 configured to provide, to a further device, an output signal responsive to or comprising the determined one or more parameters. In some examples, the further device is a user interface 290. The processing system may be further configured to display, at a user interface, a visual representation of the determined one or more parameters. Thus, the output signal provided by the output interface may control or define a display provided by the user interface.

In such case the output signal may for example represent the anatomical model including the at least part of the medical catheter system to have it shown on the display. Other suitable examples of a further device include a data storage system (e.g. memory), a further processing system, an alerting system, and so on.

The system shown in FIG. 2 is an example. Since any one of the catheter systems as disclosed herein may be used in such system, there may be any number of inputs according to the number of electrodes on a catheter system that needs to be or is desired to be connected to the processing system.

The more detailed operation of a system of FIG. 2 will now be explained.

The external electrodes 270 electrodes may comprise a plurality of electrodes angled with respect to one another (e.g. positioned orthogonally to one another), so that any electric fields generated by the electrodes are angled with respect to one another. Preferably there are three pairs of electrodes each pair having electrodes on opposing sides of the subject being investigated such as front and back, neck and lower abdomen and left and right with the region of interest being in between all pairs of electrodes. The external electrodes may also comprise a reference electrode (not shown), for example positioned on one of the legs of the subject.

The external electrodes may be controlled to generate electrical fields of different frequencies (and in different directions) by injecting current into the subject. The control of the external electrodes may be performed by the processing system 250. The electric field caused in the region of interest can be sensed by the electrodes on a catheter located within the region of interest and results in an internal electrode (i.e. “electrode”) having a different response to the externally applied electric fields based on its relative position within the subject. This information can be used to reconstruct a model of the anatomy explored with the catheter and/or track a relative location of the internal electrode within the subject and/or derive other characteristics of the electrode and/or the surrounding tissue.

For instance, if there are three external electrodes 271, 272, 273 or pairs of external electrodes positioned to emit electric fields of different frequencies (E₁, E₂, E₃) that are angled (e.g. near-orthogonal) with respect to one another, a voltage response (V₁, V₂, V₃) of an internal (intrabody) electrode (e.g. of a catheter) (e.g. identifying a voltage between the electrode and the reference electrode or between the electrode and the electrode generating the electric field) will differ depending upon position within the anatomical cavity. Other forms of response, such as an impedance response or a capacitive response (e.g. indicating change in impedance/capacitance between the internal electrode and each external electrode) will be apparent to the skilled person. These responses are dependent upon dielectric properties of surrounding tissue and/or the position of the electrode with respect to the electric fields.

Thus, a response to an electric field may comprise a voltage response, an impedance response, a capacitor response and so on. Preferably, the response is a voltage response.

The present disclosure recognizes that the response of electrodes (the electrodes) of the medical catheter system (e.g puncturing device) to such externally provided electric fields can be processed to provide useful information for aiding the performance of a tissue puncturing procedure.

It has previously been described how electrical responses of the medical catheter system can be processed by the processor circuit of the processing system to determine the one or more parameters of the medical catheter system and/or surrounding tissue of the anatomical structure. In some examples, these parameters include an anatomical model of the anatomical structure.

Thus, the responses of the electrodes of the medical catheter system can be used to perform an imaging process of the anatomical structure, thereby generating an anatomical model. Such imaged anatomical structure and model thus may include the tissue to be punctured. Since such puncturing process often is a delicate process that needs high precision regarding location and direction of puncturing such as for example is the case with transseptal puncture procedure to access the LA of the heart from the RA, having the ability to image the RA including the Inter Atrial Septum (IAS) possibly having the fossa ovalis (FO) located therein and without having to use any X-ray or US based techniques is beneficial.

The medical catheter systems disclosed herein and their use in combination with a dielectric imaging system as described herein allows such advantageous generation of an anatomical model of a structure using the catheter system as disclosed. Furthermore it allows tracking a location of the medical catheter system or parts thereof within the so determined anatomical model to guide the puncture process. In particular, the precisely defined electrode distance (or distances if there are more than two electrodes) between electrodes that have a mutually fixed location on a single part of the catheter system can provide the correct translation of sensed sets of electrode responses into locations of the electrodes in a coordinate system from which in turn the anatomical model including the IAS may be constructed and the location of the electrodes and therefrom a location and/or an orientation of at least a part of the medical catheter system relative to the anatomical model and the IAS can be generated. Thus, the anatomical model reconstruction requires the use of well-defined constant distances between electrodes and hence, the inventors have recognized that a catheter system to be used for a puncturing action should have at least one single part that includes at least two electrodes with mutually fixed location. Preferably there are more, but at least two are needed. Since a medical catheter system for puncturing tissue generally includes several components such as for example a sheath and needle or a sheath, dilator and needle, two electrodes on at least one of such components would suffice to provide the advantage.

Thus, the catheter systems as disclosed herein enable a single puncturing system to perform both anatomical imaging of the anatomical structure and to perform the actual puncturing process (e.g. by subsequently tracking the direction/position of parts of the catheter system such as for example the needle).

Use of electrodes mounted on the sheath and or the dilator if present in the catheter system perform the imaging process is particularly advantageous, as it allows the needle to be retracted inside the sheath during the imaging process for improved patient safety. It would obviate the need for having to introduce an electrophysiological catheter or imaging catheter as known in the art to be inserted through the lumen of the sheath, or dilator if present, to perform imaging. Although possible, such would increase procedural complexity as such catheter would have to be replaced with the needle after imaging.

An example imaging process is hereafter described, but it not exclusive for use with electrodes of the herein described medical catheter systems (e.g. puncturing device). Rather, any suitable internal electrodes could be used when performing a generic imaging process, such as electrodes mounted on a catheter.

To perform an imaging process, the response of the internal electrodes (e.g. to externally applied electric fields) may be iteratively recorded. The processor circuit of the processing system can apply a transfer function (“V₂R function”) that transforms each recorded response to Euclidian coordinates (R-space), whilst ensuring known properties (e.g. electrode spacing and electrical weight length) as well as a set of other constraints are maintained.

In this way, an R-space cloud of points (known Euclidian co-ordinates) can be built up and updated as the internal electrodes are moved within the anatomical cavity. Using the updated R-space cloud of points, a reconstruction algorithm generates an anatomical model of the anatomical cavity. The anatomical model may, for example, be a 3D surface that depicts or models the (bounds of) the anatomical cavity.

By way of example only, such an imaging method with all its aspects is disclosed in WO2019/034944 which is incorporated by reference. It is for this transformation that the fixed electrodes with well-defined distances provide a first advantage.

The anatomical model may be output to a display or user interface 290. The display or user interface may be configured to provide a visual representation of the anatomical model.

The process of reconstructing an anatomical model from a point cloud is conceptually illustrated in FIG. 3, which demonstrates a process 350 in which a cloud of R-space points 310 (“point cloud”) is transformed into an anatomical model 320. In the illustrated example, this is performed by creating a (3D) surface from the point cloud data, methods of which will be readily apparent to the skilled person.

For example, a point cloud can be converted into a polygon mesh or triangular mesh model (or other surface model) using a surface reconstruction approach. A variety of suitable approaches is discussed in Berger, Matthew, et al. “A survey of surface reconstruction from point clouds.” Computer Graphics Forum. Vol. 36. No. 1. 2017.

More precise identification of the bounds and features of the anatomical model can be performed by monitoring the response of the electrodes of the puncturing device (here: treated as internal electrodes) to local field measurements (e.g. fields generated by other internal electrodes) or through additional processing of global field measurements.

For example, regions with inherently marked steep gradients in the electrical field can be identified. It is recognized that such regions indicate the bounds of the anatomical cavity and/or other information, e.g. drainage of vessels into or out of a cardiac chamber as well as the valves of a cardiac chamber. These features are being picked up uniquely by the system and imaged even without physically visiting them with the catheter.

As another example, changes in a local electrical field (between two internal electrodes) can indicate the presence or absence of tissue between the two internal electrodes. A response of an internal electrode to a local electric field can therefore be used to identify the presence or absence of tissue. The processing system may itself be configured to control the local electric fields, or this can be controlled by another device in communication with the electrodes of the puncturing device. Such use of local field measurements has been described in e.g. WO WO2019/034944.

The combined global and local field measurements enable sophisticated detection and effective handling of inconsistencies and outliers, level of electrode shielding/coverage (e.g. by measuring location inter-correlation), pacing (saturation), as well as physiological drift. Drift can, for example, be detected using a moving window over time and corrected continuously whereby the catheter location remains accurate throughout the whole procedure making the system resilient to drift.

Other approaches for using external electrodes (e.g. patch electrodes) and internal electrodes (e.g. catheter electrodes) to map body volumes and visualize the locations of catheters within the map can be found in, for example, U.S. Pat. No. 10,278,616, titled “Systems and Methods for Tracking an Intrabody Catheter,” filed May 12, 2015, and U.S. Pat. No. 5,983,126, titled “Catheter Location System and Method,” filed Aug. 1, 1997, the entireties of which are hereby incorporated by reference.

In some examples, the responses of internal electrodes (or the anatomical model) may be processed to identify a target anatomical feature for identifying a location to perform a penetrating process. For example, if the penetrating process is a transseptal procedure, the target anatomical feature may be the fossa ovalis (FO) or tissue that shows the presence of a Patent Foramen Ovale. Other suitable target anatomical features will be apparent, depending upon the penetrating process.

In some examples, the responses of internal electrodes (or the anatomical model) may be processed to identify and localize areas near target anatomical features, for example, to identify potential danger areas for a penetrating process. As one example, for a transseptal process, the responses (or the anatomical model) may be processed to identify and localize the Aorta behind the IAS. This information can be used by a clinician to avoid inadvertent puncturing of the Aorta while advancing the needle across the IAS.

From the foregoing, it will be apparent that one example of a parameter of the puncturing device and/or surrounding tissue of the anatomical structure is an anatomical model of the anatomical structure (i.e. a shape/structure of the surrounding tissue). Another example parameter is a position of a target anatomical feature.

The processing system may be configured to (render and) display, at a user interface, a visual representation of the anatomical model. The visual representation of an anatomical model may, for example, comprise a 3D or flattened (panoramic) view of the anatomical structure.

Another example of a parameter of the puncturing device and/or surrounding tissue of the anatomical structure is a (relative) position of the puncturing device or particular electrodes of the puncturing device.

In particular, a relative position of the puncturing device with respect to an anatomical model (e.g. generated using the previously described imaging process) could be determined. Once an anatomical model/map of the anatomical structure has been generated by monitoring the response(s) of electrodes about the anatomical structure to electric fields, the relative position of an electrode with respect to the anatomical model/map can be readily determined by correlating a response of the electrode to a particular position.

Thus, embodiments of the present disclosure propose to further use the processing system to track a location of one or more electrodes (of another catheter) with respect to the anatomical model, i.e. to act as an electrode tracking system.

It will be apparent that, once an anatomical model of an anatomical cavity has been constructed using responses of internal electrodes within the anatomical cavity, the location of internal electrodes with respect to the anatomical model can be readily defined. For example, the transfer function used to generate the anatomical map, e.g. the “V2R function” that transforms a response to Euclidian coordinates, can be used to calculate/predict a relative position of an electrode of the puncturing device using the response of the internal electrode. The locations of the electrodes in combination with the shape and design of the catheter system allow reconstruction of a shape, location and orientation of at least the distal end of the catheter, as that end contains the electrodes.

The catheter systems as disclosed herein may also be used in a configuration where they are not used to model, but only to track the location and orientation of the distal end of the catheter. In such case, the input interface 251 of the processing system 250 may be further configured to obtain, at a fourth input (not shown) to receive or obtain from a modelling system, or imaging system or memory, an anatomical model of the anatomical structure. However, for simplification and accuracy of procedure the anatomical model is preferably generated by monitoring the location of one or more electrodes mounted upon an intraluminal device, e.g. the puncturing device or another intraluminal device such as an electrophysiology catheter that can be temporarily housed by the dilator lumen or sheath lumen before exchanging it with a needle.

The processor circuit 252 of the processing system 250 may be configured to determine the relative location of one or more electrodes of the puncturing device with respect to an anatomical model. The relative location of the one or more electrodes can be derived from various combination of the first, second and third sets of the responses. The anatomical model may, itself, be generated by monitoring the response of internal electrodes to an electric field, e.g. using the approach previously described.

In particular, the first set of one or more responses may be used to track a location of the distal end of the needle, and the second set of one or more responses may be used to track a location of the sheath or the overall puncturing device. The present disclosure recognizes that information on both of these two features can contribute to improved monitoring of the penetrating process.

This process facilitates visualization of the electrodes of the puncturing device with respect to the anatomical structure.

The processing system may therefore be further configured to display, at a user interface, a visual representation of the relative location of the (electrodes of the) puncturing device. This can be displayed with respect to a visual representation of the anatomical model, e.g. obtained at the user interface.

Although preferable, when tracking/monitoring the location of electrodes of the puncturing device with respect to an anatomical model, it is not essential that this anatomical model be generated using the puncturing device, rather a different set of internal electrodes (e.g. mounted on a catheter) could be used. Of course, a combination of both approaches could be used, i.e. the puncturing device can contribute to the mapping of the anatomical structure.

Tracking the location of electrodes of the puncturing device advantageously allows a clinician to visualize and perform a full penetrating procedure (e.g. a transseptal process) solely under dielectric sensing & imaging, i.e. without fluoroscopy or ultrasound guidance.

In particular, the maneuvering of the puncturing device to a desired location can be performed by tracking the location of the sheath (which can cover the needle to shield other tissue from the needle) and the penetrating process itself can be tracked by tracking the distal end of the needle (e.g. to track the process of the needle penetrating tissue).

Once a suitable location for performing the penetrating process has been identified, the location can be marked (e.g. with respect to the anatomical model). This can aid in future investigation of the area, e.g. for reuse of the puncturing in subsequent procedures, or in case puncturing is to be performed by another device.

Another example of a parameter of the puncturing device and/or surrounding tissue is a distance that the needle extends out of the sheath. Thus, the processor circuit may be configured to process a combination of the first second and third sets of responses to determine a distance that the needle extends out of the sheath

For example, if the relative position of the electrode(s) on the sheath and the relative position of the distal end of the needle is known, then the distance between the electrode(s) and the distal end of the needle can be calculated. This information can be used to derive/measure the distance that the needle extends out of the sheath, e.g. if the relationship between the electrode(s) and the end of the sheath is known.

As another example, the difference in the electrical responses of the electrode(s) mounted on the sheath and the distal end of the needle may be directly correlated to a distance between the electrode(s) and the distal end of the needle and/or a distance that the needle extends out of the sheath.

This information can be used to check whether or not the needle is safely covered by the sheath, e.g. during transport of the needle towards a site for penetrating tissue.

Another example of a parameter of the puncturing device and/or surrounding tissue is an orientation of the puncturing device. In particular, if the (3D) positions of two parts of the puncturing device with respect to an anatomical model are known, and the relationship between these two parts is predetermined (i.e. due to a known structure of the puncturing device), then the relative orientation of puncturing device with respect to the anatomical model can also be derived.

This information can be used to improve the display of a puncturing device by a user interface, e.g. with respect to a displayed anatomical model. Information on the orientation of the puncturing device aids a user to understand how the manipulation of the puncturing device can be performed, to correctly orient the puncturing device during transportation to a puncturing location or during the puncturing location itself.

In some examples, the orientation information and location information is used to provide a visual representation of the puncturing device with respect to a displayed anatomical model (e.g. which is correctly oriented and located within 3D space).

As yet another example, one parameter of the puncturing device and/or surrounding tissue may be an orientation/direction of the needle. In particular, the processing system may track the location of the first electrode on the sheath and the needle (using approaches previously described). As the positional relationship between these elements is predetermined, a direction of the needle can be derived (e.g. a direction of the needle may be defined as spanning between the location of the first electrode and the location of the needle).

This information can be used to improve the display of the needle by the user interface, in particular by providing an indication of the direction of the needle, e.g. alongside the displayed anatomical model to facilitate improved performance of the transseptal procedure. This is achieved as the determined direction aids a clinician in performing the transseptal procedure more accurately, by providing an indication of the direction orientation of the needle (to ensure that it is penetrating the correct area of the anatomical structure).

It has previously been described how the second electrode mounted on the sheath may be/comprise an anchoring structure 130 mounted on the sheath 110. The processor circuit of the processing system 250 may use the third set of one or more electrical responses of the anchoring structure to electric fields induced in the anatomical structure to derive one or more parameters of the puncturing device and/or surrounding tissue of the anatomical structure.

As an example, only the processor circuit of the processing system may use the third set of one or more electrical responses to track a location of the anchoring structure mounted on the sheath. Of course, the tracked location may be displayed via a user interface.

Tracking the location of this anchoring structure also aids in the performance of the penetrating process, as it provides useful information to the clinician. In particular, it can aid a clinician to understand a real-time position of the anchoring structure to thereby appropriately control the brace or support provided to the sheath, through maneuvering the anchoring structure to a particular location for bracing against the tissue, to improve an accuracy in performing a penetrating process.

As the geometric shape of the anchoring structure can be known, it is also possible to display a visual representation of the geometric shape with respect to the determined location of the anchoring structure. For example, the anchoring structure may be shaped as a loop (for bracing the sheath against tissue), which can be displayed with respect to an anatomical model. Displaying the geometric shape of the anchoring structure aids in improving a clinician's understanding of the characteristics of the puncturing device and the puncturing process.

The anchoring structure may be moveable with respect to the sheath. In particular, the anchoring structure may be able to extend and retract with respect to the sheath. The manner in which the anchoring structure moves may be known, e.g. due to known mechanics of the puncturing device. By tracking the location of the anchoring structure (using the third set of responses) with respect to the location of the first electrode on the sheath, the extent to which (i.e. the protruding distance) the anchoring structure extends from the sheath can be determined, as can the shape of the anchoring structure as it extends (due to predetermined, estimated or known device characteristics). This information can be visually represented.

As an example, the anchoring structure may comprise a loop that can be extended out of the sheath. The loop will move accordingly with a preconfigured or known loop's motion. Since the mechanical behavior of the extended loop is known, and the position of the loop is being tracked, the loop itself can also be displayed in real-time.

Previous examples of one or more parameters of puncturing device and/or surrounding tissue of the anatomical structure generally rely upon the response of electrodes of the puncturing device to externally provided electric fields. However, some parameters of the puncturing device and/or surrounding tissue of the anatomical structure may be derived from an electrical response of the electrodes of the puncturing device to internally created electric fields (e.g. electric fields generated by an electrode).

In some examples, the processor circuit may be configured to further control one or more electric fields generated by one or more of the electrodes of the puncturing device. This can be performed by controlling a current supplied to the electrode(s), e.g. over a same electric pathway used to measure an electrical response or a dedicated channel. The first, second and third sets of responses may include an electrical response to this internally generated electric field.

Yet another example of a parameter of the puncturing device and/or surrounding tissue of the anatomical structure is a measure of contact or contact force between the puncturing device and the surrounding tissue of the anatomical structure. This can be performed, for example, using the approach described in the European Patent Application having publication number EP 3294174 A1, with the electrodes of the puncturing device being treated as electrodes.

A further example of a parameter of the puncturing device and/or surrounding tissue of the anatomical structure is a thickness of tissue in the vicinity of the electrodes of the puncturing device. It is recognized that the thickness of tissue affects the dielectric properties of tissue. This, in turn, affects the electrical response of electrodes in the vicinity of the tissue. It is therefore possible to correlate information about an electrical response of electrically responsive tissue to a thickness of tissue in the vicinity of the puncturing device. As one example, a rate of change of an electrical response may be influenced by the thickness of nearby tissue, meaning that a thickness of nearby tissue can be derived by monitoring a rate of change of the electrical response. As another example, the electrical response may be directly correlated to a tissue thickness (e.g. based on predetermined calibration data).

The skilled person would be readily capable of identifying and determining other suitable examples of a parameter of the puncturing device and/or surrounding tissue of the anatomical structure using the electrical responses of the electrodes of the puncturing device.

An understanding of the usefulness of obtaining one or more parameters of the puncturing device and/or surrounding tissue of the anatomical structure using the electrical response(s) of the distal end of the needle, and two electrodes on the sheath (of which one is optionally an anchoring structure mounted on the sheath) will be hereafter described. In particular, tracking the location of these electrodes of the puncturing device (with respect to an anatomical model) provides useful information for performing a penetrating process.

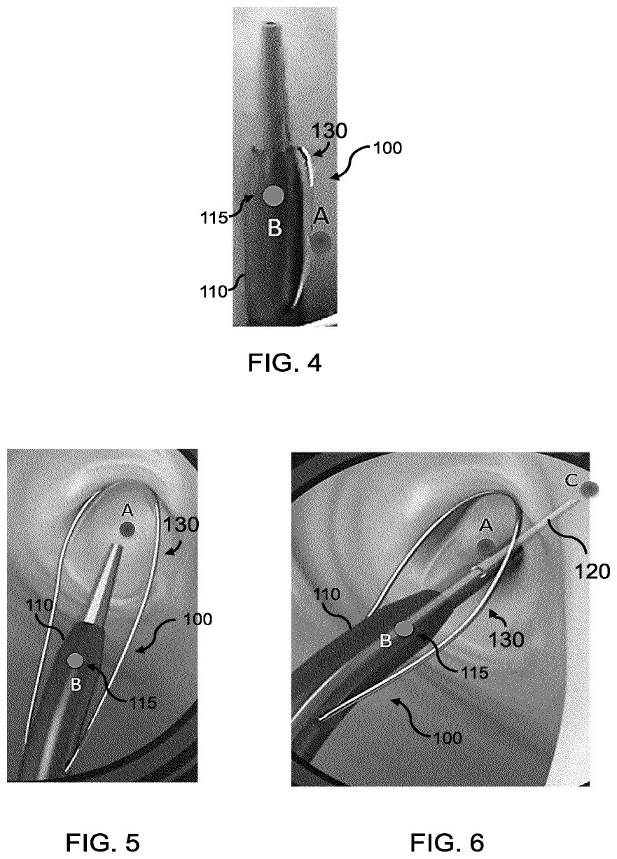

FIGS. 4, 5 and 6 illustrate a puncturing device 100 in various stages of a penetrating process, which are used to illustrate the usefulness of tracking the position/location of certain electrodes of the puncturing device throughout the penetrating process. In particular, by tracking the position of the three identified electrodes of the puncturing device, the full penetrating process can be readily monitored.

Initially, the puncturing device may be configured as illustrated in FIG. 4, in which the needle (not visible) is covered by the sheath 120.

Point A represents the spatial location of the anchoring structure's 130 (here: a loop of metal) electrical weight center and will move according to predetermined mechanic properties of the puncturing device. Point B represents the position of the first electrode 115 on the sheath.

As the puncturing device moves in the anatomical structure (e.g. to reach a target location), the electrical response(s) of the first electrode 115 on the sheath and the (non-extended) anchoring structure 130 mounted on the sheath can be monitored, e.g. to track a location/orientation of the puncturing device 100 and/or to perform an imaging process.

Thus, the electrical response of electrodes of the puncturing device can be used to derive one or more parameters of the puncturing device and/or the anatomical structure, e.g. to generate an anatomical model of the anatomical structure.

The position of a desired anatomical feature or site for performing a penetrating process can be identified (e.g. from the generated anatomical model). For instance, for a transseptal procedure, the position of the fossa ovalis could be (automatically or manually) identified from the anatomical model.

After identifying a location at which a penetrating process is to be performed, the anchoring structure 130 may be extended out of the sheath 110 to brace or anchor the sheath against the tissue (in the vicinity of the anchoring structure). Since the mechanical behavior of the anchoring structure is known, a visual representation of the shape of the anchoring structure can also be displayed in real-time by monitoring the location of the anchoring structure 130.

This results in a puncturing device being configured as illustrated in FIG. 5.

The sheath 110 can thereafter be rotated and deflected (by using the anchoring structure 130 as a brace) against the tissue) toward the desired anatomical site. The movement of the sheath can be monitored by tracking the location of the first electrode 115 on the sheath relative to the (now fixed) location of the anchoring structure 130. The movement of the sheath may cause the distal end of the sheath to displace or deform tissue of the anatomical structure (known as “tenting”). This displacement can be visually represented.

In particular, as the bounds of the anatomical structure are known (e.g. from the anatomical model) and the relationship between the first electrode 115 and the distal end of the sheath is known, it is possible to predict when the distal end of the sheath deforms the tissue of the anatomical structure (e.g. comes into contact with the bounds of the anatomical structure) by monitoring the location of the first electrode 115 with respect to the anatomical model.

The needle 120 can then be advanced through the sheath 110 (i.e. extend outwardly from a distal end of the sheath 110) to puncture the tissue with which the distal end of the sheath makes contact, as illustrated in FIG. 6. The progress of the needle during the penetrating process can be tracked by monitoring the location of the distal end 125 of the needle, as indicated by point C of FIG. 6.

The tracked location of the distal end 125 of the needle may be used, together with the tracked location of the first electrode 115, to track an orientation and/or direction of the needle. This provides useful clinical information to the clinician for ensuring that the needle is correctly positioned in a desired puncturing direction.

Of course, the electrical response of the needle may also be used before this final stage of the penetrating process. In particular, if the needle is near the distal end of the sheath (e.g. close, but not protruding from the sheath), then it will still respond to electric fields. This information can be used to improve a tracking of the puncturing device during transport to the area for performing the puncturing (e.g. to derive an orientation or shape of the puncturing device), performing an imaging process (e.g. acting as another internal electrode for the imaging process) or to determine any other suitable parameter of the puncturing device and/or surrounding tissue of the anatomical structure.

It has previously been explained how an anatomical model can be constructed using the response of internal electrodes to an electric field. This anatomical model can be rendered and displayed, e.g. at a user interface.

All of the particular characteristics of the puncturing process as described with reference to FIGS. 4, 5 and 6 such as for example the “tenting” may also be provided by use of catheter systems not having the anchoring structure.

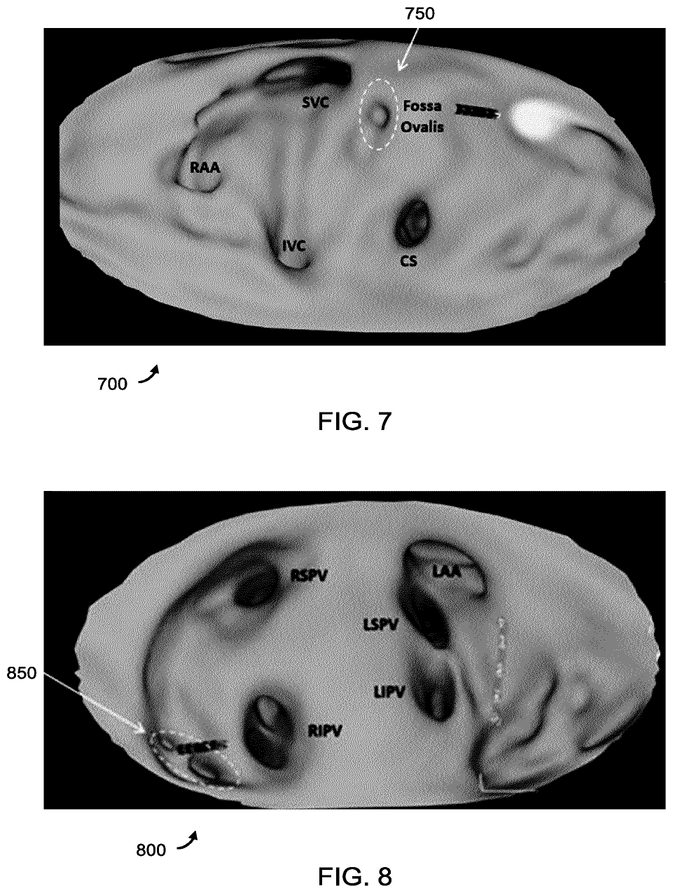

FIGS. 7 and 8 respectively depict a first 700 and second 800 representation of a rendered anatomical model. A position 750 of the fossa ovalis is identifiable from the anatomical model. These figures also illustrate the position 850 of double transseptal tracts.

The figures also illustrate the position of a penetrating device, which is obtained by tracking the location and orientation of the penetrating device using a previously described approach, and providing a visual representation of the puncturing device with respect to the anatomical model.

The advantages of the present invention, for improving an ease of performing a transseptal procedure, are apparent from the representative images provided by FIGS. 7 and 8. In particular, by providing a visual representation of the anatomy and the puncturing device, the clinician is aided in the performance of a surgical procedure by their tracking and monitoring of the relative location of the puncturing device.

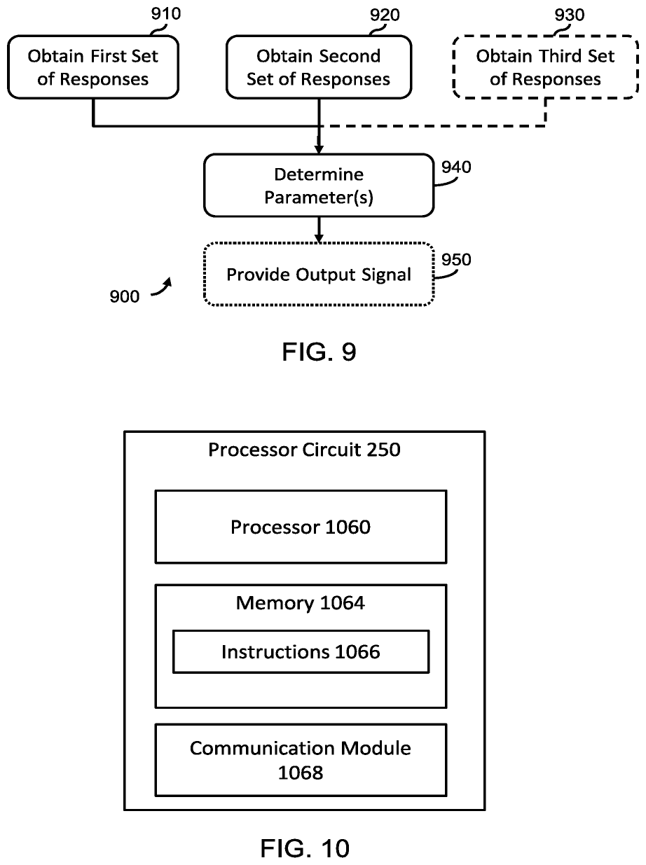

FIG. 9 is a flowchart illustrating a computer-implemented method 900 for determining one or more parameters of a puncturing device as disclosed herein.

Step 910 represents receiving or obtaining, from a second electrode mounted on the sheath, a first set of one or more electrical responses of the first electrode to one or more electrical fields induced in the anatomical structure.

Step 920 represents receiving or obtaining, from a second electrode mounted on the sheath, a second set of one or more electrical responses of the electrode mounted on the sheath to one or more electrical fields induced in the anatomical structure.

Step 930, which is optional, represents receiving or obtaining, from the distal end of the needle, a third set of one or more electrical responses of the distal end of the needle to one or more electrical fields induced in the anatomical structure, wherein the distal end of the needle is formed of a material responsive to changes in an electric field.

The computer-implemented method 900 comprises a step 940 of determining one or more parameters of the puncturing device and/or surrounding tissue of the anatomical structure using at least the first set of one or more electrical responses, the second set of one or more electrical responses and the third set of one or more electrical responses.

The method 900 may further comprise a step 950 of providing, to a further device, an output signal responsive to the determined one or more parameters of the puncturing device and/or surrounding tissue of the anatomical structure.

The one or more parameters of the puncturing device and/or surrounding tissue may be a relative position of the distal end of the needle and/or an orientation of the needle with respect to the anatomical structure.

FIG. 10 is a schematic diagram of a processor circuit 252, according to embodiments of the present disclosure. As shown, the processor circuit 250 may include a (data) processor 1060, a memory 1064, and a communication module 1068. These elements may be in direct or indirect communication with each other, for example via one or more buses. The data processor may be an analog or digital processor and preferably is a digital processor.

The processor 1060 may include a central processing unit (CPU), a digital signal processor (DSP), an ASIC, a controller, an FPGA, another hardware device, a firmware device, or any combination thereof configured to perform the operations described herein. The processor 1060 may also be implemented as a combination of computing devices, e.g., a combination of a DSP and a microprocessor, a plurality of microprocessors, one or more microprocessors in conjunction with a DSP core, or any other such configuration. In some embodiments, the processor is a distributed processing system, e.g. formed of a set of distributed processors.

The memory 1064 may include a cache memory (e.g., a cache memory of the processor 1060), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory. In an embodiment, the memory 1064 includes a non-transitory computer-readable medium. The non-transitory computer-readable medium may store instructions. For example, the memory 1064, or non-transitory computer-readable medium may have program code recorded thereon, the program code including instructions for causing the processor circuit 252, or one or more components of the processor circuit 252, particularly the processor 1060, to perform the operations described herein. For example, the processor circuit 252 can execute operations of the method 700. Instructions 1066 may also be referred to as code or program code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements. The memory 1064, with the code recorded thereon, may be referred to as a computer program product.

The communication module 1068 can include any electronic circuitry and/or logic circuitry to facilitate direct or indirect communication of data between the processor circuit 252, the puncturing device and/or the user interface (or other further device). In that regard, the communication module 1068 can be an input/output (I/O) device. In some instances, the communication module 1068 facilitates direct or indirect communication between various elements of the processing circuit 252 and/or the system (FIG. 2).

In particular, the communication module 1068 may comprise the first input and the second input for obtaining the first and second inputs respectively. The communication module 1068 may also comprise an output for providing the output signal responsive to the determined one or more parameters of the puncturing device and/or surrounding tissue of the anatomical structure.

It will be understood that disclosed methods of processing sets of responses of electrodes comprise or are preferably computer-implemented methods. As such, there is also proposed the concept of a computer program comprising computer program code for implementing any described method when said program is nm on a processing system, such as a computer or a set of distributed processors.

Different portions, lines or blocks of code of a computer program according to an embodiment may be executed by a processing system or computer to perform any herein described method. In some alternative implementations, the functions noted in the block diagram(s) or flow chart(s) may occur out of the order noted in the figures. For example, two blocks shown in succession may, in fact, be executed substantially concurrently, or the blocks may sometimes be executed in the reverse order, depending upon the functionality involved.

The present disclosure proposes a computer program (product) comprising instructions which, when the program is executed by a computer or processing system, cause the computer or processing system to carry out (the steps of) any herein described method. The computer program (product) may be stored on a non-transitory computer readable medium.

Similarly, there is also proposed a computer-readable (storage) medium comprising instructions which, when executed by a computer or processing system, cause the computer or processing system to carry out (the steps of) any herein described method. There is also proposed computer-readable data carrier having stored there on the computer program (product) previously described. There is also proposed a data carrier signal carrying the computer program (product) previously described.

The computer-readable program may execute entirely on a single computer/processor, partly on the computer/processor, as a stand-alone software package, partly on the computer/processor and partly on a remote computer or entirely on the remote computer or server (e.g. using a distributed processor processing system). In the latter scenario, the remote computer may be connected to the computer/processor through any type of network, including a local area network (LAN) or a wide area network (WAN), or the connection may be made to an external computer (for example, through the Internet using an Internet Service Provider).

It is also herein proposed to provide a processing system for determining one or more parameters of a puncturing device, comprising a sheath that houses a needle having a distal end for penetrating tissue of an anatomical structure, and/or parameters of surrounding tissue of the anatomical structure, wherein the processing system comprises: an input interface configured to obtain: at a first input, and from an anchoring structure mounted on the sheath, a first set of one or more electrical responses of the anchoring structure mounted on the sheath to one or more electrical fields induced in the anatomical structure, wherein the anchoring structure is configured to secure or couple the sheath to tissue during a penetrating procedure of the puncturing device; and a processor circuit configured to determine one or more parameters of the puncturing device and/or surrounding tissue of the anatomical structure using at least the first set of one or more electrical responses.

Thus, it is also proposed to not necessary require the distal end of the needle to act as an electrode. Rather, information may be obtained by treating an anchoring structure used to brace the sheath against tissue as an electrode. Thus, embodiments of the invention recognize an advantage in treating an anchoring structure used for bracing the sheath against tissue as an electrode, such as reduced need for alternative electrodes and improved monitoring of a penetrating process.

This processing system may be adapted wherein the input interface is configured to obtain at a second input, and from an electrode mounted on the sheath, a second set of or more electrical responses of the electrode mounted on the sheath to one or more electrical fields induced in the anatomical structure, and the processor circuit is configured to determine one or more parameters of the puncturing device and/or surrounding tissue of the anatomical structure further using at least the second set of one or more electrical responses.

Variations to the disclosed embodiments can be understood and effected by those skilled in the art in practicing the claimed invention, from a study of the drawings, the disclosure and the appended claims. In the claims, the word “comprising” does not exclude other elements or steps, and the indefinite article “a” or “an” does not exclude a plurality. A single processor or other unit may fulfill the functions of several items recited in the claims. The mere fact that certain measures are recited in mutually different dependent claims does not indicate that a combination of these measures cannot be used to advantage. A computer program may be stored/distributed on a suitable medium, such as an optical storage medium or a solid-state medium supplied together with or as part of other hardware, but may also be distributed in other forms, such as via the Internet or other wired or wireless telecommunication systems. If the term “adapted to” is used in the claims or description, it is noted the term “adapted to” is intended to be equivalent to the term “configured to”. Any reference signs in the claims should not be construed as limiting the scope.

The following examples relate to further aspects of the disclosure, but can also be used independently used from other features defined herein.

Example 1. A processing system (250) for determining one or more parameters of a puncturing device (100), comprising a sheath (110) that houses a needle (120) having a distal end (125) for penetrating tissue of an anatomical structure, and/or parameters of surrounding tissue of the anatomical structure, the processing system comprises:
an input interface (251) configured to obtain (910, 920, 930):
at a first input (251A), and from the distal end of the needle, a first set of one or more electrical responses of the distal end of the needle to one or more electrical fields induced in the anatomical structure, wherein the distal end of the needle is formed of a material responsive to changes in an electric field;
at a second input (251B), and from a first electrode (115) mounted on the sheath, a second set of or more electrical responses of the first electrode to one or more electrical fields induced in the anatomical structure; and
at a third input (251C), and from a second electrode (130, 135) mounted on the sheath, a third set of one or more electrical responses of the second electrode to one or more electrical fields induced in the anatomical structure; and
a processor circuit (252) configured to determine (940) one or more parameters of the puncturing device and/or surrounding tissue of the anatomical structure using at least the first set of one or more electrical responses, the second set of one or more electrical responses and the third set of one or more electrical responses.

Example 2. The processing system of example 1, further comprising an output interface configured to provide, to a further device, an output signal responsive to the determined one or more parameters of the puncturing device and/or surrounding tissue of the anatomical structure.

Example 3. The processing system of example 1 or 2, wherein the processing system is further configured to display, at a user interface, a visual representation of the determined one or more parameters of the puncturing device and/or surrounding tissue.

Example 4. The processing system of any of example 1 to 3, wherein the one or more parameters of the puncturing device and/or surrounding tissue is a relative position of the sheath and/or the needle with respect to the anatomical structure.

Example 5. The processing system of any of example 1 to 4, wherein:
the input interface is further configured to obtain, at a fourth input and from a modelling system or memory, an anatomical model of the anatomical structure, and
wherein the one or more parameters of the puncturing device and/or surrounding tissue comprises a relative location of the sheath and/or needle with respect to the anatomical model of the anatomical structure.

Example 6. The processing system of example 5, wherein the anatomical model of the anatomical structure is generated by monitoring the location of one or more electrodes mounted upon an intraluminal device.

Example 7. The processing system of any of example 1 to 6, wherein:
the first or second electrode comprises an anchoring structure configured to secure or couple the sheath to tissue during a penetrating procedure of the puncturing device.

Example 8. The processing system of example 7, wherein the anchoring structure is moveable relative to other components of the sheath.

Example 9. A medical system for penetrating tissue of a subject, the medical system comprising:
a puncturing device comprising:
a needle having a distal end for penetrating tissue of an anatomical structure, the distal end of the needle being formed of a first material responsive to changes in an electric field;
a sheath that houses the needle;
an electrode mounted on the sheath; and
the processing system of any of examples 1 to 8.

Example 10. The medical system of example 9, wherein the distal end of the needle is configured to puncture the inter-atrial septum of a subject.

Example 11. The medical system of any of example 8 to 10, comprising one or more external electrodes, to be positioned externally to the subject, controllable to generate one or more electric fields, wherein the distal end of the needle is configured to be responsive to changes in the one or more electric fields generated by the one or more external electrodes.

Example 12. A computer-implemented method (900) for determining one or more parameters of a puncturing device (100), comprising a sheath (110) that houses a needle (120) having a distal end (125) for penetrating tissue of an anatomical structure, and/or surrounding tissue of the anatomical structure, the computer-implemented method comprising:
obtaining (910), from the distal end of the needle, a first set of one or more electrical responses of the distal end of the needle to one or more electrical fields induced in the anatomical structure, wherein the distal end of the needle is formed of a material responsive to changes in an electric field;
obtaining (920), from a first electrode (115) mounted on the sheath, a second set of or more electrical responses of the first electrode to one or more electrical fields induced in the anatomical structure;
obtaining (930), from a second electrode (130, 135) mounted on the sheath, a third set of one or more electrical responses of the second electrode to one or more electrical fields induced in the anatomical structure; and
determining (940) one or more parameters of the puncturing device and/or surrounding tissue of the anatomical structure using at least the first set of one or more electrical responses and the second set of one or more electrical responses.

Example 13. The computer-implemented method of example 12, further comprising providing (950), to a further device, an output signal responsive to the determined one or more parameters of the puncturing device and/or surrounding tissue of the anatomical structure.

Example 14. The computer-implemented method of example 13, wherein the one or more parameters of the puncturing device and/or surrounding tissue is a relative position of the distal end of the needle with respect to the anatomical structure.

Example 15. A computer program product comprising instructions which, when executed by a suitable computer or processing system, cause the computer to carry out the method of any of examples 12 to 14.

1. A catheter for use in a tissue puncturing procedure, the catheter having a tubular body having an axial length, a distal end and a lumen extending from an opening at the distal end along at least part of the axial length, the lumen being configured to slidably house a tissue puncturing device such that the tissue puncturing device can be extended outside of the catheter through the opening and beyond the distal end of the catheter or be retracted within the catheter through the opening by a user,
wherein the catheter comprises:
a first electrode on the distal end;
a second electrode on the distal end; and
the first electrode is spaced apart from the second electrode with a fixed electrode distance, wherein, when the electrodes are in use and located within a region of interest of a subject, the first and second electrodes are capable of sensing electrical signals caused by electric fields applied to the region of interest.

2. A catheter as claimed in claim 1, wherein the tubular body includes a tapered portion at the distal end and at least one of the first and second electrodes within the tapered portion.

3. A catheter as claimed in claim 1 wherein at least the first and second electrodes are spaced apart along at least the axial length direction.

4. A catheter as claimed in claim 1, wherein the catheter is a dilator.

5. A catheter as claimed in claim 1 wherein the catheter comprises means configured to steer at least the distal end of the catheter.

6. A catheter system comprising:
a catheter as claimed in claim 1; and
at least one of:
an introducer catheter having a tubular member with a cavity therein extending between an opening at its distal end and an opening at a proximal end the tubular member, the cavity being configured to slidably house the catheter; and
the tissue puncturing device wherein the tissue puncturing device includes a distal tip operable as a third electrode.

7. A catheter system as claimed in claim 6, wherein the introducer catheter comprises means for steering at least the distal end of the introducer catheter.

8. A catheter system as claimed in claim 6, wherein the introducer catheter comprises:
a fourth electrode on the distal end;
a fifth electrode on the distal end; and
the fourth electrode is spaced apart from the fifth electrode with a fixed electrode distance, wherein, when the catheter system is in use and the fourth and fifth electrodes are located within a region of interest of a subject, the fourth and fifth electrodes are capable of sensing electrical signals caused by electrical fields applied to the region of interest.

9. A catheter as claimed in claim 1 wherein the catheter is an introducer catheter having a tubular member with a cavity therein extending between an opening at its distal end and an opening at a proximal end the tubular member, the cavity being configured to slidably house a further catheter, such as for example a dilator, the introducer catheter comprising the first and second electrodes on the distal end of the introducer catheter.

10. A catheter as claimed in claim 9 wherein the introducer catheter comprises steering means configured to steer at least the distal end.

11. A catheter system comprising:
a catheter as claimed in claim 9; and
further comprising at least one of:
a dilator including a tubular body having an axial length and a distal end and a dilator lumen extending from an opening at the distal end along at least part of the axial length, the dilator lumen being configured to slidably house a tissue puncturing device such that the tissue puncturing device can be extended outside of the dilator and beyond the distal end or be retracted within the dilator by a user; and
the tissue puncturing device, wherein the tissue puncturing device includes a distal tip operable as a third electrode.

12. A catheter as or catheter system comprising an introducer catheter wherein the introducer catheter comprises an anchoring structure at the distal end of the introducer catheter which anchoring structure is reversibly extendible from the introducer catheter and the anchoring structure comprises at least one further electrode.

13. A processing system for determining one or more parameters of tissue of an anatomical structure within a region of interest using electrical responses received or obtained from electrodes of a catheter and/or introducer catheter as claimed in claim 1, the processing system comprising:
an input interface connectable to at least first and second, or third and fourth electrodes, of the catheter and/or introducer catheter, the input interface configured to receive or obtain at least:
first electrical responses representative of electric fields induced within the region of interest; and
second electrical responses to the electric fields, wherein the first and second electrical responses are respectively received or obtained from the first and second electrodes respectively, or the fourth and fifth electrodes respectively;
a processor circuit configured to determine the one or more parameters based on at least the first and second electrical responses; and
optionally, an output configured to output data representative of the one or more parameters of the puncturing device and/or surrounding tissue of the anatomical structure based on at least the first and second electrical responses.

14. A processing system as claimed in claim 13, wherein the first electrical responses comprise first and third electrical responses representative of electric fields induced within the region of interest and the second electrical responses comprise second and fourth responses representative of electric fields induced within the region of interest, wherein the first and second electrical responses are received or obtained from the first and second electrodes, respectively, and the fourth and fifth electrical responses are received or obtained from the fourth and fifth electrodes respectively and the; and
a processor circuit configured to determine the one or more parameters based on at least the first, second, fourth and fifth electrical responses.

15. The processing system of claim 1, wherein the one or more parameters comprises an anatomical representation of the anatomical structure.

16. The processing system of claim 1, wherein the processor circuit is further configured to:
receive from a look-up table calibration data comprising for each fixed electrode distance one or more correlations between the fixed electrode distance and parameters representing electric fields measured at the electrodes spaced by the fixed electrode distances; and
determine an anatomical representation of the anatomical structure from the first and second data using the calibration data.

17. A computer-implemented method for determining one or more parameters of tissue of an anatomical structure within a region of interest using electrical responses received or obtained from electrodes of a catheter or introducer catheter as claimed in claim 1, the computer-implemented method comprising:
receiving or obtaining, by a processing system:
first electrical responses representative of electric fields induced within the region of interest; and
second electrical responses to the electric fields, wherein the first and second electrical responses are respectively received or obtained from the first and second electrodes respectively, or the fourth and fifth electrodes respectively.
determining, by a processor circuit of the processing system, the one or more parameters based on the first and second electrical responses; and
optionally, providing data representative of the one or more parameters of tissue of the anatomical structure based on at least the first and second electrical responses.

18. Computer implemented method as claimed in claim 17, wherein the one or more parameters comprises an anatomical representation of the anatomical structure.

19. Computer implemented method as claimed in claim 17, wherein the processor circuit is further configured to:
receive from a look-up table calibration data comprising for each fixed electrode distance one or more correlations between the fixed electrode distance and parameters representing electric fields measured at the electrodes spaced by the fixed electrode distances; and
the determining of the one or more parameters of tissue of an anatomical structure within a region of interest is further based on the calibration data.

20. A computer program product comprising instructions which, when executed by a processing system, cause the processing system to carry out the computer implemented method of claim 17.
